(19) United States
(12) Patent Application Publication
(10) Pub. No.: US 2023/0058505 A1
(43) Pub. Date: Feb. 23, 2023

(54) OPHTHALMIC PINHOLE PROSTHETIC WITH SURFACE MODIFICATIONS AND METHOD OF FABRICATION

(71) Applicant: Michael Snyder, Cincinnati, OH (US)

(72) Inventor: Michael Snyder, Cincinnati, OH (US)

(21) Appl. No.: 17/405,586

(22) Filed: Aug. 18, 2021

(51) Int. Cl. A61F 2/14; B33Y 80/00

(52) U.S. Cl. CPC A61F 2/15; B33Y 80/00; A61F 2220/0008; A61F 2240/002; A61F 2230/0006

(57) ABSTRACT

An ophthalmic pinhole prosthetic and a method of fabricating the same. The ophthalmic pinhole prosthetic comprises an annular portion, an inner perimeter, and an interior light transmitting portion. The annular portion may be optically opaque or at least partially optically opaque. The inner perimeter may include surface modifications configured to reduce or substantially eliminate diffraction of light compared to an ophthalmic pinhole prosthetic without the surface modifications. The interior light transmitting portion may be located within the annular portion and function to allow passage of light to interact with a retina.

### FIELD

The present teachings generally relate to an ophthalmic pinhole prosthetic and a method of fabricating an ophthalmic pinhole prosthetic. The ophthalmic pinhole prosthetic may comprise surface modifications that may be particularly advantageous to reduce or eliminate uniform diffraction of light compared to an ophthalmic pinhole prosthetic without the surface modifications.

### BACKGROUND

Focusing elements of the eye, such as the cornea and lens, generally determine how light interacts with the retina. Focusing elements of an ophthalmic patient may be abnormal (e.g., misshapen or scarring presented thereon). Abnormal focusing elements can cause at least some light rays entering the eye to converge at a focal point that is located anterior to a retina, posterior to a retina, or both. Light rays converging at focal points not coincident with the retina or fovea typically results in a visual picture with one or more anomalies, such as blur, distortion, glare, halo effect, or starburst.

Light rays that converge at a focal point anterior to the retina can occur if an eye is abnormally long or the cornea is abnormally steep; a medical condition conventionally termed as myopia. Light rays that converge at a focal point posterior to the retina may occur if an eye is abnormally short or the cornea is abnormally flat; a medical condition conventionally termed as hyperopia. A cornea may also be asymmetric or toric, resulting in an uncompensated cylindrical refractive error conventionally referred to as corneal astigmatism. Regular astigmatism can be corrected with spectacle lenses or intraocular implant lenses which contain a compensatory toric component. Irregular astigmatism, however, cannot be corrected with either spectacle lenses or intraocular implant lenses.

A normally functioning eye is typically capable of selectively focusing on either near or far objects through a process known as accommodation. Accommodation is achieved by physiologically inducing deformation in the lens via ciliary muscles. Some ophthalmic patients may have a lack of ciliary muscle control, a medical condition conventionally termed as presbyopia, which can result in light rays converging at focal points not coincident with the retina.

In order to address one or more of the medical conditions discussed above, one conventional approach involves implanting an annular mask with an interior light transmitting portion within the eye. The annular mask blocks some light rays that would otherwise pass through the focusing elements, while the interior light transmitting portion allows light rays to pass therethrough in what is referred to an a “pinhole” effect. Pinhole implants are generally described in the publication, Peter Choyce, Intra-ocular Lenses and Implants, H. K. Lewis & Co. LTD, p. 25 (1964), incorporated herein by reference for all purposes, where they are referred to as “stenopeic implants” therein.

Employing an interior light transmitting portion is discussed in U.S. Pat. No. 10,869,752 B2, incorporated herein by reference for all purposes. However, the patent teaches that the interior light transmitting portion produces a so-called “halo effect” where the patient's visual picture includes a shimmering image around objects being viewed. This phenomenon becomes more pronounced with brighter objects in a visual picture, such as light sources. The solution proposed therein involves providing the profile of the interior light transmitting portion in a geometric shape, alternative to a circular shape, which reduces the circular halo effect.

Employing an interior light transmitting portion is also discussed in U.S. Patent Application Publication No. 2014/0379078 A1, incorporated herein by reference for all purposes. The publication also teaches that patients' visual pictures include a halo or glare. It was postulated therein that the halo or glare was produced by the interior light transmitting portion. The solution proposed therein involves rounding edges of the interior light transmitting portion.

Employing an interior light transmitting portion is also discussed in U.S. Pat. No. 10,765,508, incorporated herein by reference for all purposes. The patent teaches that patients' visual pictures include a halo effect. It was postulated therein that the halo effect was produced by a plurality of holes extending through the mask region of a pinhole device placed into the cornea, the plurality of holes being provided to facilitate nutrient transport. The solution proposed therein involves varying the hole size, shape, and orientation of a substantial number of holes either randomly or otherwise non-uniformly.

It would be desirable to provide an ophthalmic pinhole prosthetic that prevents or at least substantially reduces perceived visual effects (e.g., halo effect) resulting from abnormal focusing elements of the eye. It would be desirable to provide an ophthalmic pinhole prosthetic that prevents or at least substantially reduces the amount of light that passes through damaged or irregular potions of focusing elements (e.g., cornea and lens). It would be desirable to provide an ophthalmic pinhole prosthetic that prevents or at least substantially reduces visual aberrations such as glare, halo effect, and starburst, in the visual picture of ophthalmic patients. It would be desirable to provide an ophthalmic pinhole prosthetic that prevents or at least substantially reduces uniform diffraction of light interacting with the interior light transmitting portion of the ophthalmic pinhole prosthetic. It would be desirable to provide an ophthalmic pinhole prosthetic that realizes an alternative solution to diffraction of light as a result of the light interacting with the interior light transmitting portion of the ophthalmic pinhole prosthetic.

### SUMMARY

The present disclosure relates to an ophthalmic pinhole prosthetic, which may address at least some of the needs identified above, the ophthalmic pinhole prosthetic may comprise: an annular portion that is optically opaque or at least partially optically opaque. The annular portion may comprise an inner perimeter, outer perimeter, and interior light transmitting portion. The inner perimeter may include surface modifications configured to reduce or substantially eliminate the visually apparent effects of uniform diffraction of light from the inner perimeter compared to an ophthalmic pinhole prosthetic without the surface modifications. The interior light transmitting portion may be located within the annular portion. The interior light transmitting portion may function as a passage for light to interact with a retina.

The inner perimeter may circumscribe the interior light transmitting portion. The inner perimeter may extend between an anterior surface and a posterior surface of the ophthalmic pinhole prosthetic. A length of the inner perimeter, as measured between the anterior surface and the posterior surface, may be between about 0.05 mm and 1 mm. The inner perimeter may slope from the anterior surface to the posterior surface. The inner perimeter may include a curvature. The inner perimeter may be convex or concave with respect to the interior light transmitting portion.

The interior light transmitting portion may be an aperture or a portion of optically transparent material. A diameter of the interior light transmitting portion may be between about 0.05 mm and 3 mm.

The ophthalmic prosthetic may comprise one or more surface modifications on or proximate to the inner perimeter. The one or more surface modifications may include one or more colors, one or more patterns of shapes, one or more surface topographies, one or more opacity gradients, or any combination thereof.

The surface modifications may be between about 0.1 μm to 1000 μm, more preferably between about 0.5 μm to 500 μm, or even more preferably between about 1 μm to 100 μm in their largest dimension.

The ophthalmic pinhole prosthetic may be insertable into an eye through an incision no greater than 3 mm in the largest dimension of the incision. The largest dimension may be the length between two opposing ends of the incision.

The ophthalmic pinhole prosthetic may be flexible, foldable, or both. The ophthalmic pinhole prosthetic may be rolled into a roll having an outermost diameter no greater than 3 mm.

The ophthalmic pinhole prosthetic may comprise one or more haptics. The one or more haptics may be configured to prevent the ophthalmic pinhole prosthetic from moving or rotating within an eye. The one or more haptics may be elongate projections that extend radially from the outer perimeter of the ophthalmic pinhole prosthetic. The shape of the one or more haptics may be generally C-shaped, J-shaped, plate-shaped, or any combination thereof.

The anterior surface of the ophthalmic pinhole prosthetic may be convex, and the posterior surface of the ophthalmic pinhole prosthetic may be concave; or both the anterior surface of the ophthalmic pinhole prosthetic and the posterior surface of the ophthalmic pinhole prosthetic may be generally planar.

A thickness of the ophthalmic pinhole prosthetic, as measured between the anterior surface and the posterior surface may be between about 0.05 mm and 0.8 mm. The thickness may be generally uniform, vary radially from the inner perimeter to the outer perimeter, or both.

The ophthalmic pinhole prosthetic may further comprise an outer perimeter that is radially distanced from the inner perimeter.

The present disclosure relates to a method of fabricating an ophthalmic pinhole prosthetic, which may address at least some of the needs identified above. The method may comprise locating an ophthalmic pinhole prosthetic substrate onto a support surface. The method may comprise optionally locating a template in relation to the ophthalmic pinhole prosthetic substrate. The template may have a plurality of masking regions and a plurality of pass-through regions. The method may comprise performing material addition and/or material removal on the ophthalmic pinhole prosthetic substrate to fabricate surface modifications.

The method may comprise fabricating surface modifications concurrent with performing material addition and/or material removal to realize the final dimensions of the ophthalmic pinhole prosthetic.

The surface modifications may include one or more colors, one or more patterns of shapes, one or more surface topographies, one or more opacity gradients, or any combination thereof. The surface modifications may include one or more colors, one or more patterns of shapes, one or more surface topographies, one or more opacity gradients, or any combination thereof. The one or more surface modifications may be located on or proximate to the inner perimeter.

### BRIEF DESCRIPTION OF THE DRAWINGS

FIG. 1 is a plan view of an eye.

FIG. 2 is a bi-sectional view of the eye illustrated in FIG. 1.

FIG. 3A is a perspective view of an ophthalmic pinhole prosthetic according to the present disclosure.

FIG. 3B is a plan view of an ophthalmic pinhole prosthetic according to the present disclosure.

FIG. 4A is a bi-sectional view of the ophthalmic pinhole prosthetic illustrated in FIG. 3A along line A-A.

FIG. 4B is a bi-sectional view of the ophthalmic pinhole prosthetic illustrated in FIG. 3A along line A-A.

FIG. 4C is a bi-sectional view of the ophthalmic pinhole prosthetic illustrated in FIG. 3A along line A-A.

FIG. 4D is a bi-sectional view of the ophthalmic pinhole prosthetic illustrated in FIG. 3A along line A-A.

FIG. 5A is a plan view of an inner perimeter.

FIG. 5B is a plan view of an inner perimeter.

FIG. 5C is a plan view of an inner perimeter.

FIG. 6A is a bi-sectional view of the eye illustrated in FIG. 1 with an ophthalmic pinhole prosthetic according to the present disclosure implanted therein.

FIG. 6B is a bi-sectional view of the eye illustrated in FIG. 1 with an ophthalmic pinhole prosthetic according to the present disclosure implanted therein.

FIG. 7A is a sectional view of an inner perimeter illustrated in FIG. 4A along line B-B.

FIG. 7B illustrates an ophthalmic pinhole prosthetic 40 and magnified views of an inner perimeter.

FIG. 8 is a plan view of an ophthalmic pinhole prosthetic according to the present disclosure.

### DETAILED DESCRIPTION

The present teachings may meet one or more of the above needs by the improved ophthalmic pinhole prosthetic and method of fabricating an ophthalmic pinhole prosthetic described herein. The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

The present disclosure provides for an ophthalmic pinhole prosthetic and a method of fabricating an ophthalmic pinhole prosthetic. The ophthalmic pinhole prosthetic may be an artificially fabricated device. The ophthalmic pinhole prosthetic may be implanted in one or both eyes of a living being. The ophthalmic pinhole prosthetic may be implanted in a human.

The ophthalmic pinhole prosthetic may be employed to correct the vision of a living being. The ophthalmic pinhole prosthetic may prevent or at least substantially reduce the amount of light that passes through one or more damaged and/or irregular portions (i.e., abnormal portions) of focusing elements (e.g., cornea, pupil, lens, and/or intraocular implant lens). Substantially reduce, as referred to herein, may mean reducing light transmission by about 80% or more, 85% or more, 90% or more, 95% or more, or even 99% or more. The ophthalmic pinhole prosthetic may collimate light. The ophthalmic pinhole prosthetic may be employed to prevent or at least substantially reduce visual aberrations (e.g., glare, halo effect, or starburst) in the visual picture of a patient resulting from abnormal focusing elements of the eye. Visual picture, as referred to herein, may include any object, animate being, scenery, or otherwise that reflects light that travels into the eye through the center of gaze, near-peripheral vision, mid-peripheral vision, and/or far-peripheral vison, interacts with the retina, and is converted into an electro-chemical signals that are relayed to the vision center of the brain. In other words, a visual picture is what a living being sees. The ophthalmic pinhole prosthetic may prevent or at least substantially reduce uniform diffraction of light interacting with the interior light transmitting portion of the ophthalmic pinhole prosthetic.

One byproduct of pinhole-type prostheses is the diffraction of light that interacts with the inner perimeter of the ophthalmic pinhole prosthetic as it passes through an interior light transmitting portion (pinhole). Uniform diffraction, which the present application seeks to avoid, may direct light rays uniformly across the retina of the eye. As a result, the source of visual aberrations may form a pattern across the retina, which intensifies the presence of the aberrations in the visual picture of an individual. Uniform diffraction may result from flat or otherwise uniform surfaces of the inner perimeter surrounding the interior light transmitting portion.

The ophthalmic pinhole prosthetic of the present disclosure employs surface modifications to prevent or at least substantially reduce uniform diffraction of light while non-uniform diffraction of light may still be realized. Unlike uniform diffract, non-uniform diffraction may avoid the formation of patterns of light rays across the retina that would otherwise intensify the presence of the aberrations in the visual picture of the individual. By way of this unique technique, surface modifications may be presented in various configurations to modify the diffraction of light that interacts with the inner perimeter. As will be realized by the present disclosure, the surface modifications may be in the form of one or more colors, one or more patterns of shapes, one or more surface topographies, one or more opacity gradients, or any combination thereof. Each of these types of surface modifications may be configured in various ways by modifying at least the shapes, colors, dimensions, orientations, and/or locations thereof to modify the diffraction behavior of light.

The ophthalmic pinhole prosthetic of the present disclosure may be surgically implanted in an ophthalmic patient. The ophthalmic pinhole prosthetic may be located on, proximate to, or within a focusing element (e.g., lens or lens capsule, intraocular implant lens, and/or cornea) of an eye. The ophthalmic pinhole prosthetic may be located within the cornea, within an anterior chamber (i.e., posterior to the cornea), within a posterior chamber (i.e., between the iris and lens or intraocular implant lens), within the ciliary sulcus, within the lens or intraocular implant lens, behind the lens or intraocular implant lens, or any combination thereof. The ophthalmic pinhole prosthetic may be in contacting relationship with the cornea, iris, lens capsule, lens, intraocular implant lens, or any combination thereof. The ophthalmic pinhole prosthetic may be located passively within the eye or may be sutured to one or more anatomical structures of the eye.

An incision may be made in one or more anatomical structures of the eye to locate the ophthalmic pinhole prosthetic therein. The incision may be made in a focusing element (e.g., lens and/or cornea) of the eye. The incision may be made in plural anatomical structures in the eye to pass the ophthalmic pinhole prosthetic through the plural anatomical structures. For example, in order to locate the ophthalmic pinhole prosthetic in the anterior or posterior chamber, an incision may be formed in the cornea, the limbus, or the sclera. The ophthalmic pinhole prosthetic may be deformed to fit through an incision. The ophthalmic pinhole prosthetic may be deformed by bending, folding, rolling, compressing, dehydrating, or any combination thereof. The ophthalmic pinhole prosthetic may be un-bent, un-folded, un-rolled, un-compressed, hydrated when located within the desired location and/or orientation in the eye. The ophthalmic pinhole prosthetic may be located within an intraocular implant lens during manufacture thereof. In this manner, the ophthalmic pinhole prosthetic may be introduced into the eye concurrently with the intraocular implant lens.

The ophthalmic pinhole prosthetic may be supported in the eye by physiological structures, artificial structures surgically introduced in the eye, or both. Physiological structures may include the cornea, anterior chamber, posterior chamber, ciliary sulcus, lens, or any combination thereof. Artificial structures introduced in the eye may include haptics, sutures, or both. The physiological structures and/or artificial structures may prevent or substantially reduce movement or rotation of the ophthalmic pinhole prosthetic within the eye after the surgical implantation procedure is completed. Substantially reduce, in this context may mean radial or axial (e.g., along the optical axis) displacement of no more than 0.5 mm, more preferably 0.1 mm, or even more preferably 0.01 mm.

The ophthalmic pinhole prosthetic may include an interior light transmitting portion, as described herein. Conventionally, there are two types of ophthalmic prosthetics with an interior light transmitting portion. The two types may include iris prosthetics and pinhole implants.

Iris prosthetics may function to reduce the amount of light entering the eye. Iris prosthetics may be typically employed to treat anatomic iris defects (e.g., deformed iris) and functional iris defects (e.g., improper contraction or dilation of the iris or absence of pigment within the iris tissue). Current versions of iris prosthetics do not incorporate aperture sizes considered to deliver the pinhole optical response.

Pinhole implants may function to prevent or at least substantially reduce the amount of light that passes through abnormal focusing elements (e.g., cornea and lens). Pinhole implants may typically include one or more optically opaque portions that block light and an interior light transmitting portion, as described herein, that allows light to enter the eye. Pinhole implants are typically employed to treat abnormal focusing elements and/or increase depth of focus. The ophthalmic pinhole prosthetic of the present disclosure may be classified as a pinhole implant. Pinhole implants may be characterized by an interior light transmitting portion having a diameter of between about 0.05 mm and 3 mm. Ophthalmic pinhole prosthetics having a diameter larger than this range may not be classified as a pinhole implant. As a result, ophthalmic prosthetics having a diameter larger than this range may function differently than pinhole implants and/or may be employed to treat ophthalmic conditions that are distinct from those treated by pinhole implants.

The ophthalmic pinhole prosthetic of the present disclosure may be employed to treat abnormal corneas due to scarring, abnormal corneas due to corneal disease (e.g., keratoconus), abnormal lenses, congenital aniridia (i.e., complete or partial absence of an iris), traumatic aniridia, albinism, surgical removal of part or all of an iris (e.g., due to melanoma), or other known iris defects.

Ophthalmic Pinhole Prosthetic

The ophthalmic pinhole prosthetic of the present disclosure may function to at least partially block the passage of light rays to the retina. The ophthalmic pinhole prosthetic may block light rays that pass through abnormal focusing elements (e.g., cornea or lens). For example, a cornea may be abnormal and an ophthalmic pinhole prosthetic according to the present disclosure may be located in an anterior chamber and prevent light rays passing through the cornea from interacting with the retina. The ophthalmic pinhole prosthetic may block light rays that that would have otherwise interacted with abnormal focusing elements if the ophthalmic pinhole prosthetic were not present in the eye. For example, a lens may be abnormal and the ophthalmic pinhole prosthetic according to the present disclosure may be located in a posterior chamber and prevent light rays from interacting with abnormal portions of the lens. The ophthalmic pinhole prosthetic may reduce the amount of light entering the eye relative to an eye that is free of an ophthalmic pinhole prosthetic. The ophthalmic pinhole prosthetic may reduce day-time and/or night-time light sensitivity; reduce or substantially eliminate visual aberrations such as glare, halo effect, starburst, or any combination thereof; improve cosmetic appearance of the eye; or any combination thereof. The ophthalmic pinhole prosthetic may at least partially block some or all wavelengths of light in the visible spectrum. The ophthalmic prosthetic may at least partially block or substantially fully transmit light outside of the visible spectrum.

The ophthalmic pinhole prosthetic may be fabricated from a biocompatible material. The ophthalmic pinhole prosthetic may be fabricated from a polymer. The biocompatible polymer may include polymethylmethacrylate, polycarbonate, polyvinylidene fluoride, polyvinyl chloride, polypropylene, polyethylene, polystyrene, polyether ether ketone, polysulfone, polyimide, prolene, hydrophilic acrylic, hydrophobic acrylic, hydrogel, silicone, expanded polytetrafluoroethylene, the like, or any combination thereof.

The ophthalmic pinhole prosthetic may be defined by a thickness. The thickness may be measured between an anterior surface and posterior surface of the ophthalmic pinhole prosthetic. The anterior surface may oppose the posterior surface. The anterior surface may be oriented toward the anterior portion of the eye after implantation into a living being. The posterior surface may be oriented toward the posterior portion of the eye after implantation into a living being. The thickness may be between about 0.05 mm and 0.8 mm. The thickness may be about 0.05 mm or more, 0.1 mm or more, or even 0.2 mm or more. The thickness may be about 0.8 mm or less, 0.6 mm or less, 0.5 mm or less, or even 0.4 mm or less. The ophthalmic pinhole prosthetic may have a uniform thickness. The ophthalmic pinhole prosthetic may have a thickness that varies from an inner perimeter to an outer perimeter. The variation of thickness may be gradual. It may be continuous and/or step-wise. The thickness may have tolerances in conformance with ISO 11979-3.

The ophthalmic pinhole prosthetic may maintain its dimensional properties (e.g., diameter and/or thickness) and structural integrity (e.g., free of tearing) after implantation into a patient's eye. The ophthalmic pinhole prosthetic may maintain its dimensional properties (e.g., diameter and/or thickness) and structural integrity (e.g., free of tearing) after surgical manipulation (e.g., surgical implantation) with an intraocular lens injector and/or cartridge according to the present teachings. The ophthalmic pinhole prosthetic may maintain its dimensional properties (e.g., diameter and/or thickness) and structural integrity (e.g., free of tearing) after surgical manipulation with forceps (e.g., surgical implantation or removal) in conformance with ISO 11979-3.

The ophthalmic pinhole prosthetic may include a curvature. The curvature may correspond to a curvature of a focusing element (e.g., cornea or lens). The curvature may be customized to generally correspond to the curvature of an individual patient's eye. The curvature may contribute, at least in part, to the focusing of light rays that enter the eye. The anterior surface may be convex. The posterior surface may be concave.

The anterior surface and/or posterior surface may be generally planar. The anterior surface and posterior surface may be generally parallel to one another.

The ophthalmic pinhole prosthetic may have a generally uniform or non-uniform surface roughness on the anterior surface, posterior surface, inner perimeter, outer perimeter, or any combination thereof. As referred to herein, a generally uniform surface roughness may mean an average roughness (Sₐ) of about 10 nm or less. That is, an average of absolute values of the surface differences in a given area may be about 10 nm or less from a mean plane. Surface roughness may be measured according to the method set forth in Lewandowska et. al., The technique of measurement of intraocular lens surface roughness using Atomic Force Microscopy, Interdisciplinary Journal of Engineering Sciences, Vol. II, No. 1 (2014), incorporated herein by reference for all purposes.

The ophthalmic pinhole prosthetic may comprise a fiber portion, as described herein. An ophthalmic pinhole prosthetic that is free of a fiber portion may have a tear strength of about 30 mN or more, 50 mN or more, or even 70 mN or more. An ophthalmic pinhole prosthetic that is free of a fiber portion may have a tear strength of about 130 mN or less, 110 mN or less, or even 90 mN or less. An ophthalmic pinhole prosthetic that includes a fiber portion may have a tear strength of about 300 mN or more, 500 mN or more, or even 700 mN or more. An ophthalmic pinhole prosthetic that includes a fiber portion may have a tear strength of about 1,300 mN or less, 1,100 mN or less, or even 900 mN or less.

The ophthalmic pinhole prosthetic may be custom-fabricated for different patients. The custom fabrication may include a custom dimensions (e.g., diameter and/or thickness), custom color of the annular portion, custom number and configuration of haptics, custom profile shape (e.g., circular, elliptical, and/or ovoid) or any combination thereof. For example, the dimensions may be customized to suitably fit the dimensions of a patient's eye. The ophthalmic pinhole prosthetic may substantially mimic the color and appearance of a patient's eye without the ophthalmic pinhole prosthetic.

The ophthalmic pinhole prosthetic may be foldable, flexible, rollable, compressible, or any combination thereof. The ophthalmic pinhole prosthetic may be sufficiently foldable, flexible, rollable, and/or compressible to be insertable into an eye through an incision no greater than 3 mm in the largest dimension. As referred to herein with reference to an incision, the largest dimension may be the length between two opposing ends of the incision. The ophthalmic pinhole prosthetic may be rollable into a roll having an outermost diameter of no greater than 3 mm. The ophthalmic pinhole prosthetic may be foldable into a bi-fold, tri-fold, or even quad-fold of no greater than 3 mm in the largest dimension. The ophthalmic pinhole prosthetic may be elastic. That is, after folding, flexing, rolling, or compressing the ophthalmic pinhole prosthetic, it may return to its pre-deformed dimensions.

The ophthalmic pinhole prosthetic may be insertable into an eye by any suitable intraocular lens injector. As a non-limiting example, the intraocular lens injector may include a handpiece. The handpiece may include a plunger rod (which may be spring-loaded) with an actuator (e.g., a screw-style actuator adapted for a push and twist motion) for delivering the device (e.g., via a cartridge). One commercial example of such a device is the UNFOLDER® Silver, commercially available from Johnson & Johnson. The intraocular lens injector may be employed with a cartridge. As a non-limiting example, the cartridge may include the PSCST cartridge, commercially available from Johnson & Johnson. After introduction into the eye, the ophthalmic pinhole prosthetic may un-folded, un-flexed, un-compressed, and/or un-rolled. Elastic properties of the ophthalmic pinhole prosthetic may cause it to at least partially un-fold, un-flex, un-compress, and/or un-roll without manipulation by an ophthalmologic physician. The ophthalmic pinhole prosthetic may be manipulated by an ophthalmologic surgeon to un-fold, un-flex, un-compress, and/or un-roll the ophthalmic pinhole prosthetic. The ophthalmic pinhole prosthetic may be inserted in a deturgesced, dehydrated form and regain its original (i.e., prior to being deturgesced and dehydrated) size and physical characteristics once hydrated within the eye.

The ophthalmic pinhole prosthetic may comprise one or more annular portions, apertures, inner perimeters, surface modifications, outer perimeters, interior light transmitting portions, fiber portions, haptics, or any combination thereof.

The ophthalmic pinhole prosthetic may comprise one or more annular portions. The annular portion may function to prevent or at least substantially reduce transmission of light in the visible and/or outside the visible spectrum to the retina relative to an eye free of an ophthalmic pinhole prosthetic according to the present teachings. The annular portion may prevent or at least substantially reduce transmission of light from the visible and/or outside the visible spectrum (e.g., infrared spectrum), that passes through abnormal focusing elements (e.g., cornea or lens), to the retina relative to an eye free of an ophthalmic pinhole prosthetic according to the present teachings. For example, the annular portion may be located in an eye posterior to an abnormal focusing element and transmission of light passing through the abnormal focusing elements may be prevented or at least substantially reduced by the annular portion. The annular portion may prevent or at least substantially reduce transmission of light through abnormal focusing elements relative to an eye free of an ophthalmic pinhole prosthetic according to the present teachings. For example, the annular portion may be located in an eye anterior to an abnormal focusing element and prevent or at least substantially reduce transmission of light interacting with the abnormal focusing elements.

The annular portion may be optically opaque or at least partially optically opaque to visible light (i.e., light having a wavelength of from about 390 nm to 700 nm). As referred to herein, the term “opaque” may mean preventing or at least substantially reducing the transmission of radiant energy (e.g., light), by absorption and/or reflection of the radiant energy. As referred to herein, a material and/or structure that is opaque may have a light transmissivity of about 30% or less, more preferably 20% or less, more preferably 10% or less, or even more preferably 5% or less. The annular portion may be transparent to light in an infrared range (i.e., light having a wavelength of from about 750 nm to 1,000 μm). As referred to herein, a material and/or structure that is transparent may have a light transmissivity of about 70% or more, more preferably 80% or more, more preferably 90% or more, or even more preferably 100%.

The annular portion may have a uniform transmissivity across one or more dimensions (e.g., diameter and/or thickness). The annular portion may have two or more regions of varying transmissivity. Transmissivity may vary gradually or step-wise. For example, the transmissivity may gradually increase from the inner perimeter of the ophthalmic pinhole prosthetic to the outer perimeter of the annular portion, or vice versa. The transmissivity may vary over one or more ranges in different regions of the ophthalmic pinhole prosthetic. That is, one region may vary in transmissivity by 30%, from one end of the region to an opposing end of the region, while another region may vary in transmissivity by 10%, from one end of the region to an opposing end of the region. The transmissivity may vary over smaller ranges in regions proximate to the inner perimeter, outer perimeter, or both.

The annular portion may be a discrete structure that is located on the anterior and/or posterior surface of the ophthalmic pinhole prosthetic. The annular portion may be a discrete structure that is encapsulated within the ophthalmic pinhole prosthetic (i.e., between the anterior and posterior surface). The annular portion may be encapsulated in the ophthalmic pinhole prosthetic by a molding process, lamination, material addition process, or any combination thereof. The material addition process may be a series of continuous additions, discrete sequential additions, or both. The discrete structure may be a polymeric member or metal member (e.g., film) that is distinct from the polymer employed to fabricate the other structures of the ophthalmic pinhole prosthetic.

The annular portion may be unitary with the ophthalmic pinhole prosthetic. For example, the annular portion may comprise a region of polymer from which the ophthalmic pinhole prosthetic is fabricated, the polymer having dye and/or pigment injected thereinto and/or disposed thereon (e.g., on an anterior and/or posterior surface of the ophthalmic pinhole prosthetic).

The annular portion may be fabricated from a material (e.g., polymer) treated with dye, pigment, or both. Dyes may refer to substances soluble in solvent that are typically optically transparent and absorb but do not scatter light. Pigments may refer to powdered substance suspensions. Pigments may be generally more optically opaque than dyes. The dye, pigment, or both may be a biologically inert substance. The dye, pigment, or both may be approved by the FDA. Exemplary, non-limiting dyes and pigments may include those enumerated in 21 C.F.R. Part 73, Subpart D and 21 C.F.R. Part 74, Subpart D, incorporated herein by reference in their entirety for all purposes. The concentration of dye and/or pigment may modulate the transmissivity and/or absorption of the annular portion. The chemical identity of the dye and/or pigment may modulate the wavelengths of light absorbed by the dye and/or pigment.

Transmissivity of the annular portion may be modulated by increasing or decreasing the concentration of dyes and/or pigments, optically opaque particles, or any combination thereof.

The annular portion may be fabricated from a material (e.g., polymer) with optically opaque particulates dispersed therein. The optically opaque particulates may include carbon nanoparticles. The optically opaque particles may be dispersed in a polymer in a liquid state. The dispersion of optically opaque particles may be positionally fixed by hardening and/or cross-linking of the polymer. The optically opaque particulates may be in the form of spheres, rods, whiskers, horns, pyramids, the like, or any combination thereof. The optically opaque particulates may be about 10 nm or more, 20 nm or more, 30 nm or more, or even 40 nm or more in their largest dimension. The optically opaque particulates may be about 100 nm or less, 90 nm or less, 80 nm or less, 70 nm or less, or even 60 nm or less in their largest dimension. By way of example, the largest dimension of a rod may be the length of the rod as opposed to the diameter of the rod.

The annular portion may be fabricated from a naturally optically opaque material. The films may include carbon, metal, polymer, or any combination thereof.

The annular portion may be fabricated into a film. The film may have a thickness of about 1 μm or more, 5 μm or more, 10 μm or more, or even 20 μm or more. The film may have a thickness of about 500 μm or less, 300 μm or less, 100 μm or less, or even 50 μm or less.

One or both of a discrete structure annular portion and unitary annular portion, as taught herein, may be employed in an ophthalmic pinhole prosthetic. One or any combination of dye and/or pigment, optically opaque particulates, and naturally optically opaque materials, as taught herein, may be employed in an ophthalmic pinhole prosthetic. For example, an ophthalmic pinhole prosthetic may comprise an annular portion fabricated from both of a dye and/or pigment and optically opaque particles, both dispersed in a matrix of polymer.

An anterior surface, posterior surface, outer perimeter, and/or inner perimeter of the annular portion may be subjected to material removal, material addition, or both, according to the present teachings. Material removal and/or material addition may influence transmissivity, absorption, or reflection of the annular portion, or any combination thereof. For example, etching (e.g., chemical etching) may reduce the transmissivity of the annular portion.

The thickness of the annular portion may be generally equal to or less than the thickness of the ophthalmic pinhole prosthetic. The thickness of the annular portion may be between about 0.05 mm and 0.8 mm. The thickness of the annular portion may be about 0.05 mm or more, 0.1 mm or more, or even 0.2 mm or more. The thickness of the annular portion may be about 0.8 mm or less, 0.6 mm or less, 0.5 mm or less, or even 0.4 mm or less. Transmissivity of the annular portion may be modulated by increasing or decreasing the thickness of the annular portion, ophthalmic pinhole prosthetic, or both.

The annular portion may be defined by a diameter of a circle or length of a major axis of an oval or ellipse. The diameter or length of a major axis may be generally equal to or less than the diameter or length of a major axis of the outer perimeter. The diameter or length of a major axis may be between about 1 mm and 15 mm. The diameter or length of a major axis may be about 1 mm or more, 2 mm or more, 4 mm or more, or even 6 mm or more. The diameter or length of a major axis may be about 15 mm or less, 13 mm or less, 11 mm or less, or even 9 mm or less.

The ophthalmic pinhole prosthetic may comprise one or more interior light transmitting portions. The interior light transmitting portion may function to allow passage of light to the retina and/or fovea. The interior light transmitting portion may be located within the annular portion. The interior light transmitting portion may be generally concentric with the annular portion (i.e., co-linear central axes). A central axis of the interior light transmitting portion may be offset from a central axis of the annular portion. The interior light transmitting portion may include an aperture or a portion of optically transparent material. As referred to herein, a material and/or structure that is transparent may have a light transmissivity of about 70% or more, more preferably 80% or more, more preferably 90% or more, or even more preferably 100%. The aperture may be free of material (e.g., through-hole).

The interior light transmitting portion may be defined by a central axis extending transversely through its geometric center. The central axis of the interior light transmitting portion may be coaxial with an optical axis of the eye. The central axis of the interior light transmitting portion may be offset from an optical axis of the eye. The central axis may be offset by about 0.001 mm or more, 0.005 mm or more, or even 0.01 mm or more from an optical axis of the eye. The central axis may be offset by about 1 mm or less, 0.5 mm or less, or even 0.1 mm or less from an optical axis of the eye.

The central axis of the interior light transmitting portion may be coaxial with the central axis of the pupil. The central axis of the interior light transmitting portion may be offset from the central axis of the pupil. The central axis may be offset by about 0.001 mm or more, 0.005 mm or more, or even 0.01 mm or more from the central axis of the pupil. The central axis may be offset by about 1 mm or less, 0.5 mm or less, or even 0.1 mm or less from the central axis of the pupil.

The interior light transmitting portion may be defined by a diameter of a circle or a length of a major axis of an oval or ellipse. The diameter or length of a major axis of the interior light transmitting portion may be generally equal to the diameter or length of a major axis of the inner perimeter. The diameter or length of a major axis may be between about 0.05 mm and 3 mm. The diameter or length of a major axis may be about 0.05 mm or more, 0.1 mm or more, 0.5 mm or more, or even 1 mm or more. The diameter or length of a major axis may be about 3 mm or less, 2.5 mm or less, 2 mm or less, or even 1.5 mm or less. The diameter or length of a major axis may be generally equal to, less than, or greater than a diameter of a pupil. It may be particularly advantageous for the diameter or length of a major axis to be less than the diameter of the pupil in order to restrict the amount of light entering the eye as compared to the amount allowed to pass by a pupil that is unobstructed by an ophthalmic pinhole prosthetic according to the present teachings.

The ophthalmic pinhole prosthetic may include an inner perimeter. The inner perimeter may extend between an anterior surface and posterior surface of the ophthalmic pinhole prosthetic. The inner perimeter may be radially distanced from the outer perimeter of the ophthalmic pinhole prosthetic. The inner perimeter may circumscribe the interior light transmitting portion.

The inner perimeter may be generally perpendicular to the anterior surface, posterior surface, or both. The inner perimeter may slope from the anterior surface to the posterior surface, or vice versa. The slope may extend at an angle from an orthogonal axis of the anterior surface and/or posterior surface. The angle may be about 1° or more, 2° or more, 5° or more, or even 10° or more. The angle may be about 45° or less, 40° or less, 30° or less, or even 20° or less. The inner perimeter may have a curvature. The inner perimeter may be convex or concave with respect to the interior light transmitting portion.

The inner perimeter may be defined by a length, as measured between the anterior surface and posterior surface of the ophthalmic pinhole prosthetic. The length may be between about 0.1 mm and 1 mm. The length may be about 0.1 mm or more, 0.2 mm or more, or even 0.3 mm or more. The length may be about 1 mm or less, 0.8 mm or less, 0.7 mm or less, or even 0.6 mm or less.

The inner perimeter, if free of surface modifications, may generate diffraction patterns. The diffraction patterns may be uniform. The uniform diffraction patterns may be present in the visual picture of the patient as visual aberrations in the form a glare, halo effect, or starburst. The diffraction patterns may be generated by the interaction of light with the inner perimeter. The diffraction patterns may cause light rays to scatter across the retina uniformly. Scattering of light rays across the retina may produce a glare, halo effect, or starburst in the visual picture of the patient.

The inner perimeter may include one or more undulations. The undulations may be present around the entire perimeter or at least a portion thereof. The undulations may be characterized by a wave-like shape of the inner perimeter.

The present disclosure addresses uniform diffraction patterns generated by the inner perimeter by including surface modifications on or proximate to the inner perimeter.

The ophthalmic pinhole prosthetic may comprise surface modifications. The surface modifications may function to reduce or substantially eliminate uniform diffraction of light as compared to an ophthalmic pinhole prosthetic without the surface modifications of the present disclosure. The surface modifications may absorb light, reflect light internally, reflect light away from the eye, cause the spread of diffracted light across the retina, cause non-uniform diffraction, or any combination thereof. Internal reflection of light may refer to a repetitive reflection between two or more adjacent and opposingly oriented surfaces that propagates in a direction that is generally radially outward (i.e., toward the outer perimeter of the ophthalmic pinhole prosthetic). Each iteration of repetitive reflection may result in the absorption of at least a portion of the light ray by the ophthalmic pinhole prosthetic.

The surface modifications may be located on or proximate to the inner perimeter. The surface modifications may be disposed on the interior light transmitting portion proximate to the inner perimeter, the annular portion proximate to the inner perimeter, or both.

The surface modifications may be between about 0.1 μm to 1,000 μm in their largest dimension. The surface modifications may be about 0.1 μm or more, 1 μm or more, 10 μm or more, 50 μm or more, or even 100 μm or more in their largest dimension. The surface modifications may be about 1,000 μm or less, 750 μm or less, 500 μm or less, or even 250 μm or less in their largest dimension.

The largest dimension, referring to the colors, may be the length between the two most distanced points of the surface area occupied by a portion of color. For example, the largest dimension of colors disposed as dots on the surface of the ophthalmic pinhole prosthetic may be the diameter of the dot.

The largest dimension, referring to the shapes, may be the length between the two most distance points of the surface area occupied by a shape, the distance a groove extends into a surface of the ophthalmic pinhole prosthetic, the distance a ridge extends from a surface of the ophthalmic pinhole prosthetic, the distance between adjacent crests, the distance between adjacent troughs, or any combination thereof.

The largest dimension, referring to the surface topography, may be the distance a groove extends into a surface of the ophthalmic pinhole prosthetic, the distance a ridge extends from a surface of the ophthalmic pinhole prosthetic, the distance between adjacent crests, the distance between adjacent troughs, or any combination thereof.

The largest dimension, referring to the opacity gradient, may be the length of the annular region occupied by the opacity gradient from its inner diameter to its outer diameter.

The surface modifications may comprise one or more colors, one or more patterns of shapes, one or more surface topographies, one or more opacity gradients, or any combination thereof.

The surface modifications may comprise one or more colors. The colors may be characterized by a wavelength. The colors may include any color in the CMYK color space. The colors may be characterized by hue, chroma, intensity, saturation, luminance, brightness value, opacity, or any combination thereof. The colors may include two or more colors, three or more colors, four or more colors, or even five or more colors.

The color may be imparted by dye, pigment, or both. Dyes may refer to substances soluble in solvent that are typically optically transparent and absorb but do not scatter light. Pigments may refer to powdered substance suspensions. Pigments may be generally more optically opaque than dyes. The dye, pigment, or both may be a biologically inert substance. The dye, pigment, or both may be approved by the FDA. Exemplary, non-limiting dyes and pigments may include those enumerated in 21 C.F.R. Part 73, Subpart D and 21 C.F.R. Part 74, Subpart D, incorporated herein by reference for all purposes.

It may be particularly advantageous to prevent dyes and/or pigments from bleeding or leaching into the ophthalmic pinhole prosthetic or the eye. The dye and/or pigment may have a suitably high covalency to prevent bleeding or leaching. The dye and/or pigment may be surface bonded to the ophthalmic pinhole prosthetic. Surface bonding may involve providing a dye and/or pigment with a linking chemical moiety and grafting the same to the polymer of the ophthalmic pinhole prosthetic after polymerization. The dye and/or pigment may be diffused into the polymer matrix of the ophthalmic pinhole prosthetic. Dye and/or pigment may be applied to an ophthalmic pinhole prosthetic and a coating (e.g., thin film) may be located over the dye and/or pigment to prevent or at least substantially prevent bleeding or leaching.

The colors may be disposed on surfaces of the ophthalmic pinhole prosthetic in the form of one or more shapes. Typically, the colors may be in the form of dots however other shapes are contemplated by the present disclosure. The colors may be continuous across one or more surfaces of the ophthalmic pinhole prosthetic. The shapes (e.g., dots) may be produced by inkjet printing. The shape of the colors may be determined by a template, as taught herein. Inkjet printing may produce dots of dye and/or pigment having a diameter of between about 50 μm and 500 μm. The diameter may be about 50 μm or more, 100 μm or more, 150 μm or more, or even 200 μm or more. The diameter may be about 500 μm or less, 450 μm or less, 400 μm or less, 350 μm or less, or even 300 μm or less.

Two or more colors may be spaced from one another, overlap with one another, or both. Overlapping of or adjacent two or more colors may produce a color that is different from the two or more colors. For example, overlapping or adjacent red and blue may produce purple.

The colors may be selected to absorb light of one or more different wavelengths. Selectively employing colors to absorb light of particular wavelengths may modulate the diffraction pattern generated by the inner perimeter.

The surface modifications may comprise one or more patterns of shapes. The patterns of shapes may be a generally uniform pattern (i.e., uniform shape(s), orientation of shapes, and/or spacing between shapes). The shapes may include any suitable geometric shape. The shapes may include any 3-sided, 4-sided, 5-sided, 6-sided, 7-sided, or even 8-sided shape (e.g., polygon). The shapes may include an amorphous shape. It may be particularly advantageous for the shapes to include one or more amorphous shapes. An amorphous shape may be defined with a geometry that has one or more undulations, lobes, sides, corners, or any combination thereof.

The one or more patterns of shapes may be defined by grooves extending into the ophthalmic pinhole prosthetic from the inner perimeter, ridges extending from the inner perimeter, a surface area occupied by dye/pigment, or any combination thereof. The grooves, ridges, dye/pigment, or any combination thereof may be disposed around the profile of the shapes where the internal area of the shapes may be free of the same. The grooves, ridges, dye/pigment, or any combination thereof may be disposed throughout the area of the shapes.

The patterns of shapes may be apparent to a viewer from a viewing angle that is at least orthogonal to the surface of the inner perimeter.

The grooves and/or ridges may function to alter the light (e.g., light which would create a detectable image that is perceived by a patient) as compared to an inner perimeter without the grooves and/or ridges. For example, the grooves and/or ridges may function to scatter, absorb, and/or reflect light differently as compared to an inner perimeter without the grooves and/or ridges.

The one or more patterns of shapes may be fabricated as part of the inner perimeter by material removal and/or material addition, as taught herein.

The surface modifications may comprise one or more surface topographies. The surface topography may be defined by a plurality of crests and troughs formed in surfaces of the ophthalmic pinhole prosthetic and facets extending between crests and troughs. The crests and/or troughs may be curved, pointed, or both. The crests and/or troughs may extend a length from a mean plane. The mean plane may be defined by an average plane between crests and troughs. Different portions of the inner perimeter may include surface topographies, or the inner perimeter may have a uniform surface topographies over the entire inner perimeter.

The crests and troughs may project from the anterior surface to the posterior surface of the ophthalmic pinhole prosthetic. That is, individual crests and troughs may extend the length between the anterior surface to the posterior surface of the ophthalmic pinhole prosthetic. When viewed along a viewing angle orthogonal to the anterior surface or posterior surface of the ophthalmic prosthetic, the crests and troughs may both be visible to the viewer.

The crests and troughs may project perimetrically around the inner perimeter. That is, individual crests and troughs may extend the length around the inner perimeter. When viewed along a viewing angle orthogonal to the anterior surface or posterior surface of the ophthalmic prosthetic, the foremost crest and facet may be visible to the viewer.

The crests and troughs may be randomly distributed across the surface of the inner perimeter. That is, a plurality of crests and troughs may be disposed along the length between the anterior and posterior surfaces of the ophthalmic pinhole prosthetic and/or perimetrically around the inner perimeter. The randomly distributed crests and troughs may not be disposed in any discernable rows, layers, or otherwise.

The surface topography may be scalloped. Scalloped may mean crests that are curved, and troughs are pointed, with the troughs being disposed at the interface of curvature radii of adjacent crests; or troughs that are curved and crests that are pointed, the crests being disposed at the interface of curvature radii of adjacent troughs.

The surface topography may be jagged. Jagged may mean crests and troughs that are both pointed. Facets between points may be straight, curved, or both.

The surface topography may be irregular. Irregular may mean a random distribution of pointed crests, curved crests, pointed troughs, curved troughs, straight facets, curved facets, or any combination thereof.

The surface topography may be generally uniform. That is, the crests and troughs may extend a generally uniform length above and/or below a mean plane, have generally the same shape (e.g., pointed and/or curved), have generally equal distances between adjacent crests and/or troughs, or any combination thereof.

The surface topography may vary. That is, the crests and troughs may extend different lengths above and/or below a mean plane, have different shapes (e.g., pointed and/or curved), have different distances between adjacent crests and/or troughs, or any combination thereof. The crests and/or troughs may extend radially.

The surface topography may function to alter the light (e.g., light which would create a detectable image that is perceived by a patient) as compared to an inner perimeter without the surface topography. For example, the surface topography may function to scatter, absorb, and/or reflect light differently as compared to an inner perimeter without the surface topography.

The one or more surface topographies may be fabricated as part of the inner perimeter by material removal and/or material addition, as taught herein.

The one or more surface modifications may include one or more opacity gradients. The opacity gradient may decrease in opacity from the annular portion to the interior light transmitting portion. The opacity gradient may decrease in opacity from the antterior surface of the ophthalmic pinhole prosthetic to the posterior surface of the ophthalmic pinhole prosthetic or vice versa. The opacity may decrease by about 10% or more, 30% or more, or even 50% or more. The opacity may decrease by about 100% or less, 90% or less, or even 70% or less.

The opacity gradient may be located in both a region of the annular portion and a region of the interior light transmitting portion. By way of example, the opacity gradient may extend into an interior light transmitting portion that is fabricated from optically transparent material. The opacity gradient may be located in an annular gradient region located in both the annular portion and the interior light transmitting portion. The annular gradient region may be defined by a length from its inner diameter to its outer diameter. The length may be about 0.01 mm or more, 0.05 mm or more, or even 0.1 mm or more. The length may be about 1 mm or less, 0.5 mm or less, or even 0.3 mm or less.

The opacity gradient may be fabricated by varying a concentration of dye and/or pigment, a concentration of optically opaque particulates, a thickness of a film, a depth of etching, or any combination thereof. The dye and/or pigment, optically opaque particulates, film, and etching may be identified and/or applied to the ophthalmic pinhole prosthetic according to the teachings herein.

The ophthalmic pinhole prosthetic may comprise an outer perimeter. The outer perimeter may extend between an anterior surface and posterior surface of the ophthalmic pinhole prosthetic. The outer perimeter may be radially distanced from the inner perimeter.

The outer perimeter may be generally perpendicular to the anterior surface, posterior surface, or both. The outer perimeter may slope from the anterior surface to the posterior surface, or vice versa. The slope may extend at an angle from an orthogonal axis of the anterior surface and/or posterior surface. The angle may be about 1° or more, 2° or more, 5° or more, or even 10° or more. The angle may be about 45° or less, 40° or less, 30° or less, or even 20° or less. The outer perimeter may have a curvature. The outer perimeter may be flat or convex or concave with respect to the interior light transmitting portion. It may be particularly advantageous for the outer perimeter to be convex or concave.

The outer perimeter may be defined by a diameter of a circle or length of a major axis of an oval or ellipse. The diameter or length of a major axis may be between 5 mm and 15 mm. The diameter or length of a major axis may be about 5 mm or more, 10 mm or more, 10.5 mm or more, 11 mm or more, or even 11.5 mm or more. The diameter or length of a major axis may be about 15 mm or less, 14 mm or less, 13.5 mm or less, 13 mm or less, or even 12.5 mm or less.

The ophthalmic pinhole prosthetic may include one or more haptics. The haptics may function to prevent the ophthalmic pinhole prosthetic from moving or rotating within the eye. The haptics may extend radially from the outer perimeter of the ophthalmic pinhole prosthetic. The haptics may be generally elongate projections that extend radially from the outer perimeter of the ophthalmic pinhole prosthetic. The arms may extend orthogonal or at any acute angle to a tangent of the outer perimeter. Arms may curve toward and or away from the outer perimeter. The haptics may be C-shaped, J-shaped, plate-shaped, or any other suitable design.

The haptics, when implanted into a living being, may be opposed against an inner surface of an eye. For example, the haptics may oppose against a perimeter of a posterior chamber or anterior chamber. The haptics may be elastic. The diameter of elastic haptics may be larger than a diameter of an eye structure (e.g., perimeter of the anterior chamber) so that the haptics deform when located within the eye and apply pressure against the eye structure. The diameter of elastic haptics may be 0.1%, 0.5%, 1%, 2%, or even 3% larger than the diameter of an eye structure.

The haptics, when implanted into a living being, may be mounted to an eye structure via sutures. The haptics may be employed with sutures to prevent the prosthetic from moving or rotating within the eye. The haptics may include elongate projections that extend radially from the outer perimeter of the ophthalmic pinhole prosthetic.

The haptics may extend from the outer perimeter of the ophthalmic pinhole prosthetic. The haptics may extend a length radially from the outer perimeter of the ophthalmic pinhole prosthetic. The length may be between about 3 mm and 10 mm). The length may be about 3 mm or more, 4 mm or more, or even 6 mm or more. The length may be about 10 mm or less, 9 mm or less, or even 8 mm or less.

The ophthalmic pinhole prosthetic described herein may include two, three, four, or more haptics. The haptics may be located equidistant with respect to each other around the outer perimeter. The haptics may be located at different distances from each other around the outer perimeter. The haptics may be located on opposing sides of the outer perimeter.

The haptics may be co-planar with the outer perimeter. The haptics may be oriented at an angle to the plane of the outer perimeter. The angle may be about 1° or more, 3° or more, 5° or more, or even 7° or more. The angle may be about 15° or less, 13° or less, 11° or less, or even 9° or less. The haptics may be planar and/or step-vaulted.

The shape, size, orientation, and/or number of the haptics may depend on the location within the eye where the prosthetic is to be located.

Examples of suitable haptics are disclosed in U.S. Pat. Nos. 4,634,442; 5,192,319; 6,106,553; 6,228,115; and 7,455,691, which are incorporated herein by reference in their entirety for all purposes.

The ophthalmic pinhole prosthetic may include a fiber portion or may be free of a fiber portion. The fiber portion may function to increase the mechanical strength (e.g., tearing strength) of the ophthalmic pinhole prosthetic. The fiber portion may be included in the ophthalmic pinhole prosthetic where suturing the ophthalmic pinhole prosthetic is desired (e.g., suturing of haptics). The fiber portion may allow the ophthalmic pinhole prosthetic to withstand tearing or cheese-wiring imposed by suturing. An ophthalmic pinhole prosthetic that is free of a fiber portion may be suitable for suturing, however looser sutures may be necessary to prevent tearing or cheese-wiring. Cheese-wiring, as referred to herein, may mean the cutting or deformation the ophthalmic pinhole prosthetic caused by the tension of sutures.

The fiber portion may be fabricated from a polymer meshwork. The polymer may include polyester, polymethylmethacrylate, polycarbonate, polyvinylidene fluoride, polyvinyl chloride, polypropylene, polyethylene, polystyrene, polyether ether ketone, polysulfone, polyimide, prolene, hydrophilic acrylic, hydrophobic acrylic, hydrogel, silicone, expanded polytetrafluoroethylene, the like, or any combination thereof.

The fiber portion may be fabricated by material addition and/or material removal, as taught herein.

The fiber portion may be provided as one or more layers on and/or within the ophthalmic pinhole prosthetic. The fiber portion may be located on an anterior surface and/or posterior surface of the ophthalmic pinhole prosthetic. The ophthalmic pinhole prosthetic may be molded around the fiber portion. The fiber portion may be laminated with layers of the ophthalmic pinhole prosthetic. The fiber portion may be adhered to the ophthalmic pinhole prosthetic.

Fabrication

The ophthalmic pinhole prosthetic may be fabricated by material removal and/or material addition. Material removal may include milling, lathing, grinding, etching, or any combination thereof. Material addition may include molding, 3D printing, thin film deposition, spraying, brushing, rolling, swabbing, or any combination thereof.

Material removal and/or material addition may be employed to realize the final dimensions of an ophthalmic pinhole prosthetic, fabricate surface modifications, or both.

Milling and/or lathing may be performed with tooling. Examples of suitable tooling employed with lathing processes may include, but is not limited to, turning bits, facing bits, chamfering bits, boring bits, concave bits, convex bits, cutoff bits, the like, or any combination thereof. Examples of suitable tooling employed with milling processes may include, but is not limited to, square bits, ball bits, tapered bits, engraving bits, the like, or any combination thereof.

The tooling may have a surface roughness that is transferred to a surface upon its interaction with the tooling. The surface roughness of the tooling may be deliberately selected to provide the desired surface roughness. The surface roughness may be produced by deflection of the tooling and/or surface. The surface roughness may be produced by geometric error or the milling or lathing equipment.

Milling and/or lathing may be performed with micro-tooling. Micro-tooling may have a diameter of about 40 μm or more, 60 μm or more, 70 μm or more, or even 80 μm or more. Micro-tooling may have a diameter of about 160 μm or less, 140 μm or less, 120 μm or less, or even 100 μm or less.

Grinding may be performed with an abrasive substrate, abrasive compound, or both. The abrasive substrate may be a surface coated with abrasive particles. The abrasive particles may be adhered to the surface. The abrasive substrate may be a material with a roughened surface. The abrasive substrate may be ANSI (American National Standards Institute) rated 60 grit or more, 80 grit or more, 100 grit or more, 150 grit or more, or even 220 grit or more. The abrasive substrate may be ANSI rated 1200 grit or less, 800 grit or less, 500 grit or less, 360 grit or less, or even 280 grit or less. The abrasive compound may comprise a suspension of abrasive particles in a liquid medium. The liquid may be a paste. The abrasive particles may be about 5 μm or more, 10 μm or more, 50 μm or more, or even 100 μm or more in their largest dimension. The abrasive particles may be about 600 μm or less, 500 μm or less, 400 μm or less, or even 300 μm or less in their largest dimension.

Etching may be performed by exposing one or more surfaces to an acidic (e.g., hydrofluoric acid, sulfuric acid, or nitric acid) or basic (e.g., potassium hydroxide, sodium hydroxide, or tetramethylammonium hydroxide) solution. This may otherwise be referred to as chemical etching. The acid or base may be present in an aqueous solution. Chemical etching may remove material. Chemical etching may remove material from a surface by influencing the scission of polymer chains. Chemical reactions of the surface may be ceased by quenching the acidic or basic solution with a neutralizing solution. The etching may give rise to surface roughness, as described herein.

The depth of chemical etching (i.e., depth below a mean plane of a pre-etched/engraved surface, as disclosed herein) may be modulated by the exposure time, pH, acid/base concentration, temperature, or any combination thereof. The depth may be about 0.001 μm or more, 0.01 μm or more, 0.1 μm or more, or even 1 μm or more. The depth may be about 50 μm or less, 40 μm or less, 30 μm or less, 20 μm or less, or even 10 μm or less.

Etching may be performed with radiation. This may otherwise be referred to as laser etching. A laser may be employed which emits light radiation of a wavelength between about 3 μm and about 50 μm (i.e., mid-to far-infrared). Laser etching may sublimate polymer from the surface by the generation of a plasma or heat upon the surface.

The depth of laser etching (i.e., depth below a mean plane of a pre-etched/engraved surface, as disclosed herein) may be modulated by the exposure time, wavelength, intensity, or any combination thereof. The depth may be about 0.001 μm or more, 0.01 μm or more, 0.1 μm or more, or even 1 μm or more. The depth may be about 50 μm or less, 40 μm or less, 30 μm or less, 20 μm or less, or even 10 μm or less.

Molding may include injection molding, co-injection molding, and/or overmolding. Co-injection molding and/or overmolding may be particularly advantageous where the annular portion is a discrete structure from the material of the ophthalmic pinhole prosthetic. Co-injection molding and/or overmolding may be particularly advantageous where a fiber portion, as disclosed herein, is employed.

3D printing (“additive manufacturing”) may involve the deposition of material in a plurality of layers to sequentially build up the ophthalmic pinhole prosthetic. Deposition may be skipped in regions of the ophthalmic pinhole prosthetic to define grooves and/or apply material in discrete regions to define ridges. Material may be deposed as one or more continuous lengths of extrudate, jetted droplets, or both. Material may or may not be deposed onto or into a mold. The mold may include a negative impression of surface modifications (e.g., surface topography) thereon. During deposition of material, the material may be shaped by the negative impression. Upon hardening and/or curing of the material, the negative impression may be fixed in the material. Material may be removed from the 3D printed ophthalmic pinhole prosthetic to achieve desired dimensions, and/or surface roughness.

Thin film deposition may include physical vapor deposition, chemical vapor deposition, or both. Thin film deposition may provide a layer having a thickness of about 1 nm or more, 5 nm or more, 10 nm or more, or even 20 nm or more. Thin film deposition may provide a layer having a thickness of about 500 nm or less, 300 nm or less, 100 nm or less, or even 50 nm or less.

Method

The method may comprise one or more of the following steps. Some of the steps may be duplicated, removed, rearranged relative to other steps, combined into one or more steps, separated into two or more steps, or a combination thereof.

The present disclosure provides for a method of fabricating an ophthalmic pinhole prosthetic. The method may comprise locating an ophthalmic pinhole prosthetic substrate onto a support surface. The ophthalmic pinhole prosthetic substrate may be a “blank” from which a final ophthalmic pinhole prosthetic may be fabricated. The ophthalmic pinhole prosthetic substrate may comprise a polymer in accordance with the present disclosure. The ophthalmic pinhole prosthetic substrate may include an annular portion, an interior light transmitting portion, or both. The interior light transmitting portion may include an aperture or a portion of optically transparent material. The aperture may be formed into the ophthalmic pinhole prosthetic substrate by a process of material addition and/or material removal, according to the present disclosure. After fabricating surface modifications on an inner perimeter, an aperture may be filled with optically transparent material.

The ophthalmic pinhole prosthetic substrate may be formed by slicing thin wafers from an elongate bar of stock material. The thin wafers may have a thickness that is generally equal to a final desired thickness of the ophthalmic pinhole prosthetic or greater than a final desired thickness of the ophthalmic pinhole prosthetic. Where the thickness of the thin wafers is greater than a final desired thickness of the ophthalmic pinhole prosthetic, the thin wafer may be processed by material removal to realize a final desired thickness of the ophthalmic pinhole prosthetic. Curvature (e.g., concavity and convexity) may be formed into the thin wafers by material removal. Curvature (e.g., concavity and convexity) may be formed into the thin wafers by heating and cooling the thin wafers in a mold.

The bar of stock material may comprise molded or extruded polymer according to the present disclosure. Molding may include injection molding, co-injection molding, and/or overmolding. Co-injection molding and/or overmolding may be particularly advantageous to provide an annular portion that is a discrete structure from the material of the ophthalmic pinhole prosthetic. The bar of stock material may be extruded or co-extruded. Co-extrusion may be particularly advantageous to provide an annular portion that is a discrete structure from the material of the ophthalmic pinhole prosthetic.

Where haptics are desired, a radius of the bar of stock material may be generally equal to or greater than the radius of the most distanced portion of the haptics from the center of the ophthalmic pinhole prosthetic. A cross-sectional profile of the bar of stock material may include a rough shape of the desired haptic shape (e.g., C-shaped, J-shaped, or plate shaped) of the final ophthalmic pinhole prosthetic. The haptics may be formed by material removal of the rough shape. The haptics may be formed after extracting thin wafers from the bar of stock material.

The method may comprise performing material addition and/or material removal on an ophthalmic pinhole prosthetic substrate to realize the final dimensions of the ophthalmic pinhole prosthetic. Material removal may include milling, lathing, etching, or any combination thereof, according to the present teachings. Material addition may include molding, 3D printing, thin film deposition, spraying, brushing, rolling, swabbing, or any combination thereof, according to the present teachings.

The method may comprise fabricating surface modifications concurrent with performing material addition to realize the final dimensions of the ophthalmic pinhole prosthetic. For example, milling of an interior light transmitting portion may impart a surface topography on the inner perimeter. As another example, material may be deposited into a mold to form the ophthalmic pinhole prosthetic, the mold including a negative impression of surface modifications (e.g., surface topography), and upon hardening and/or curing of the material, the negative impression may be fixed in the material.

The method may optionally comprise locating an ophthalmic pinhole prosthetic substrate onto a support surface. The support surface may fixate the ophthalmic pinhole prosthetic substrate during material removal and/or material addition. The support surface may prevent movement or rotation of the ophthalmic pinhole prosthetic substrate during material removal and/or material addition.

The method may optionally comprise locating a template in relation to the ophthalmic pinhole prosthetic substrate. The template may be located a distance away from the ophthalmic pinhole prosthetic substrate, directly on an anterior or posterior surface of the ophthalmic pinhole prosthetic substrate, directly on the inner perimeter, or any combination thereof. The template may function to delineate where material removal takes place. The template may function to delineate where dye and/or pigment interacts with the inner perimeter. The template may be employed to form surface modifications. It may be particularly advantageous to employ a template when surface modifications are formed by a process of material removal (e.g., chemical or laser etching) or material addition (e.g., deposition of thin films, dyes, and/or pigments).

The template may include a plurality of masking regions, a plurality of pass-through regions, or both. The plurality of masking regions may be defined by the presence of material that covers one or more surfaces of the ophthalmic pinhole prosthetic or otherwise prevents radiation and/or physical materials (e.g., dye) from contacting one or more surfaces of the ophthalmic pinhole prosthetic. The plurality of masking regions may be optically opaque. The plurality of masking regions may be infrared opaque. The plurality of pass-through regions may comprise optically and/or infrared transparent material, apertures, or both. The plurality of pass-through regions may function to allow radiation and/or physical materials (e.g., dye) to pass through and interact with the inner perimeter.

The plurality of masking regions may prevent or at least substantially prevent radiation (e.g., infrared radiation) from interacting with one or more surfaces of the ophthalmic pinhole prosthetic substrate. The plurality of masking regions may prevent or at least substantially prevent dye and/or pigment from interacting with one or more surfaces of the ophthalmic pinhole prosthetic substrate. The plurality of masking regions may prevent or at least substantially prevent thin film deposition from interacting with one or more surfaces of the ophthalmic pinhole prosthetic substrate. The plurality of masking regions may prevent or at least substantially prevent acidic or basic solution from interacting with one or more surfaces of the ophthalmic pinhole prosthetic substrate.

The plurality of pass-through regions may allow radiation (e.g., infrared radiation) to interact with one or more surfaces of the ophthalmic pinhole prosthetic substrate. The plurality of pass-through regions may allow dye and/or pigment to interact with one or more surfaces of the ophthalmic pinhole prosthetic substrate. The plurality of pass-through regions may allow thin film deposition to interact with one or more surfaces of the ophthalmic pinhole prosthetic substrate. The plurality of pass-through regions may allow acidic or basic solution to interact with one or more surfaces of the ophthalmic pinhole prosthetic substrate.

The template may include pass-through regions in the form of one or more patterns of shapes. The template may include masking regions in the form of one or more patterns of shapes. The patterns of shapes may be realized in one or more surfaces of the ophthalmic pinhole prosthetic substrate by material addition and/or material removal, according to the present disclosure. For example, dye may be ink jetted toward the ophthalmic pinhole prosthetic substrate with a template located therebetween, the dye passing through pass-through regions and impeded by masking regions.

The method may comprise performing material addition and/or material removal to fabricate surface modifications. The surface modifications may be fabricated on an inner perimeter of the ophthalmic pinhole prosthetic substrate. Material removal may include milling, lathing, etching, or any combination thereof, according to the present teachings. Material addition may include molding, 3D printing, thin film deposition, spraying, brushing, rolling, swabbing, or any combination thereof, according to the present teachings.

The surface modifications may comprise one or more colors, one or more patterns of shapes, one or more surface topographies, or any combination thereof, according to the present teachings.

### DETAILED DESCRIPTION OF THE DRAWINGS

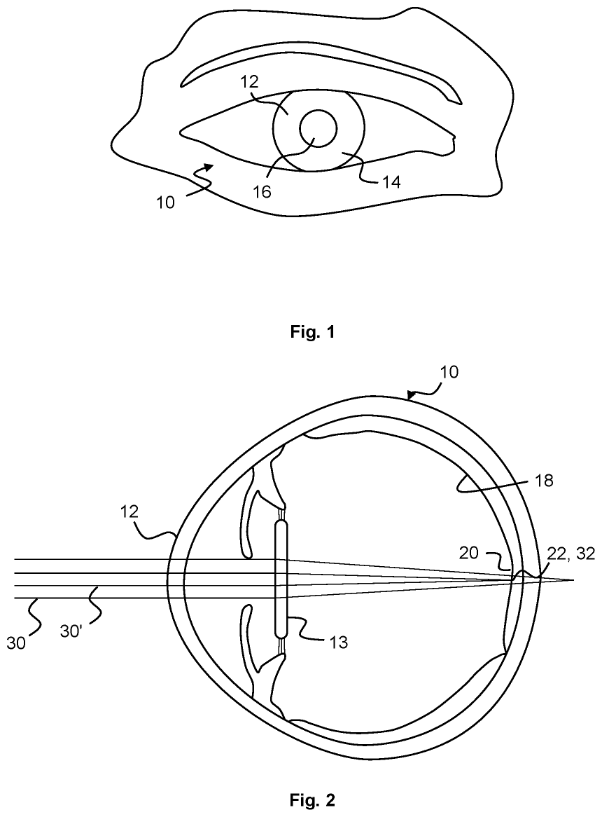

FIG. 1 is a plan view of an eye 10. The eye 10 resides in an eye socket in the skull. The eye 10 includes an annular portion of pigmented tissue known as the iris 14. The iris 14 includes smooth muscle for controlling and regulating the size of a pupil 16 located in the iris 14, pupil 16 being an opening in the iris 14. The eye 10 includes a cornea 12 that covers and protects both the iris 14 and pupil 16.

FIG. 2 is a bi-sectional view of the eye 10 illustrated in FIG. 1. The eye 10 includes a cornea 12 and intraocular lens 13 located posterior to the cornea 12. The eye 10 includes a retina 18, which lines the interior of the posterior surface of the eye 10. The retina 18 includes the receptor cells which are primarily responsible for the sense of vision. The retina 18 includes a highly sensitive region, known as the macula 20, where signals are received and transmitted to the visual centers of the brain via the optic nerve. The retina 18 includes a point with particularly high sensitivity, known as the fovea 22.

FIG. 2 illustrates the transmission of light into and through the eye 10. Due to an aberration of the cornea 12, some light rays 30 entering the eye 10 and passing through the cornea 12 and the intraocular lens 13 are refracted in such a way that the light rays 30 do not converge at a single focal point on the retina 18. Rather, the light rays 30 converge at a point posterior the retina 18. Other light rays 30′ pass through portions of the cornea 12 without aberrations and converge at a single focal point 32 that is coincident with the fovea 22.

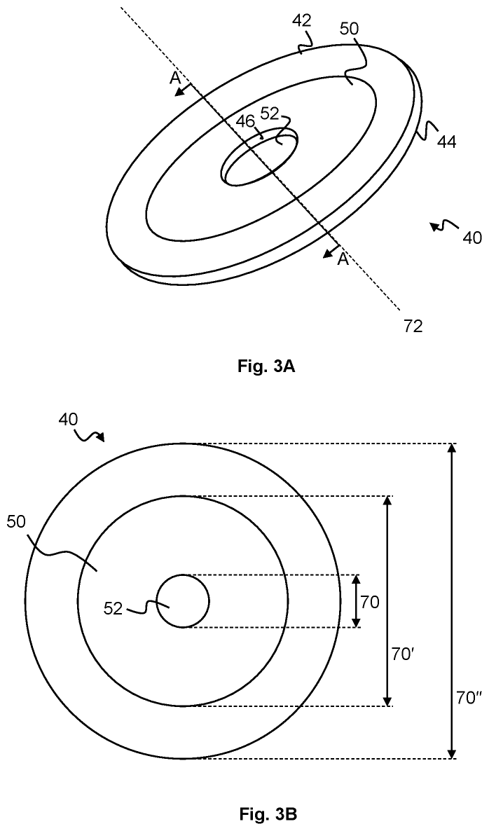

FIG. 3A is a perspective view of an ophthalmic pinhole prosthetic 40. The ophthalmic pinhole prosthetic 40 includes an anterior surface 42 adapted to orient away from the retina 18 upon implantation into an eye 10, shown in FIG. 2, and a posterior surface 44 adapted to orient toward the retina 18 upon implantation into an eye 10. The ophthalmic pinhole prosthetic 40 includes an annular portion 50. An interior light transmitting portion 52 is located centrally within the annular portion 50. The central axis 72 of the annular portion 50 is co-axial with the central axis 72 of the interior light transmitting portion 52. The interior light transmitting portion 52 is defined by an inner perimeter 46 extending between the anterior surface 42 and the posterior surface 44 of the ophthalmic pinhole prosthetic 40.

FIG. 3B is a plan view of an ophthalmic pinhole prosthetic 40. The ophthalmic pinhole prosthetic 40 includes an annular portion 50 and an interior light transmitting portion 52. The ophthalmic pinhole prosthetic 40 is defined by a diameter 70″. The annular portion 40 is defined by a diameter 70′. The interior light transmitting portion 52 is defined by a diameter 70.

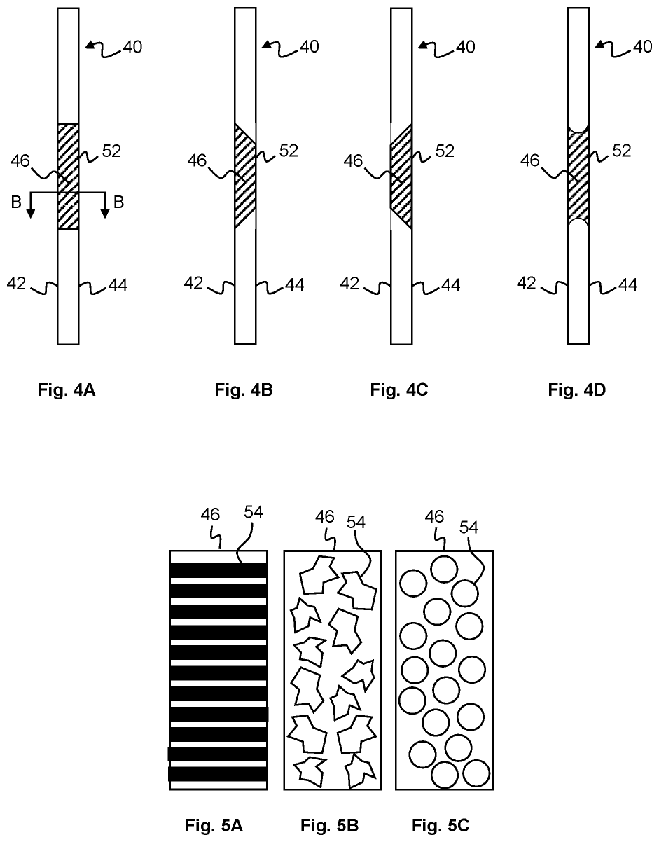

FIG. 4A through FIG. 4D are bi-sectional views of ophthalmic pinhole prosthetics 40. The ophthalmic pinhole prosthetic 40 includes an anterior surface 42 adapted to orient away from the retina 18 upon implantation into an eye 10, shown in FIG. 2, and a posterior surface 44 adapted to orient toward the retina 18 upon implantation into an eye 10. The ophthalmic pinhole prosthetic 40 includes an interior light transmitting portion 52 located centrally therein. The interior light transmitting portion 52 is defined by an inner perimeter 46 extending between the anterior surface 42 and the posterior surface 44 of the ophthalmic pinhole prosthetic 40.

FIG. 4A illustrates an inner perimeter 46 that is orthogonal to both the anterior surface 42 and posterior surface 44. FIG. 4B illustrates an inner perimeter 46 that is sloped from the posterior surface 44 to the anterior surface 42. FIG. 4C illustrates an inner perimeter 46 that is sloped from the anterior surface 42 to the posterior surface 44. FIG. 4D illustrates an inner perimeter 46 that is convex with respect to the interior light transmitting portion 52.

FIG. 5A through FIG. 5C are plan views of the inner perimeter 46. The inner perimeter 46 comprises surface modifications 54. The surface modifications 54 are patterns of shapes fabricated on the inner perimeter 46. The shapes can be fabricated by material addition and/or material removal as disclosed herein.

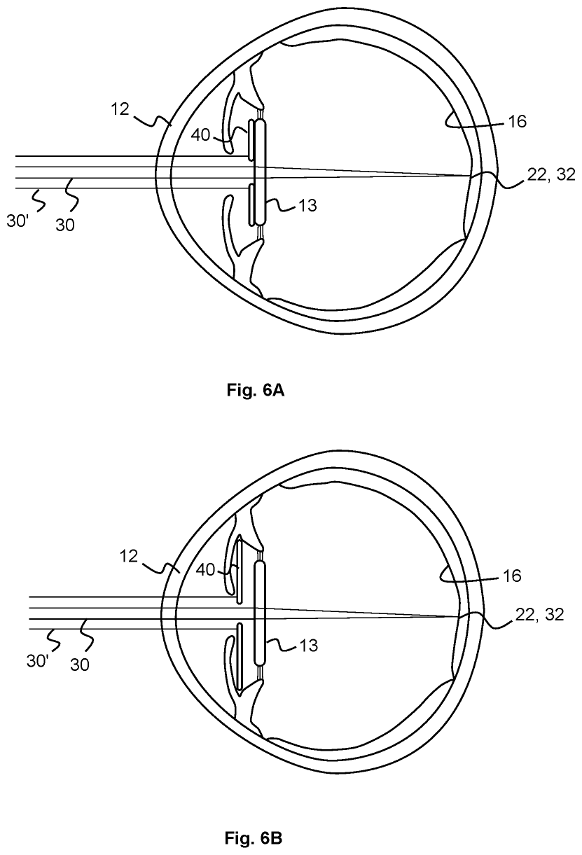

FIG. 6A and FIG. 6B are bi-sectional views of an eye 10. In FIG. 6A, the ophthalmic pinhole prosthetic 40 has been inserted into the capsular bag surrounding an intraocular lens 13. In FIG. 6B, the ophthalmic pinhole prosthetic 40 has been inserted into the anterior chamber of the eye 10 and the outer perimeter of the ophthalmic pinhole prosthetic 40 is in contact with the ciliary sulcus. Light rays 30 that pass through the ophthalmic pinhole prosthetic 40 converge at a single focal point 32 that is coincident with the fovea 22. Light rays 30′ that have passed through aberrations in the cornea 12 are blocked by the ophthalmic pinhole prosthetic 40 preventing passage of light rays 30′ that would otherwise not converge at a single focal point 32, such as what is illustrated in FIG. 2.

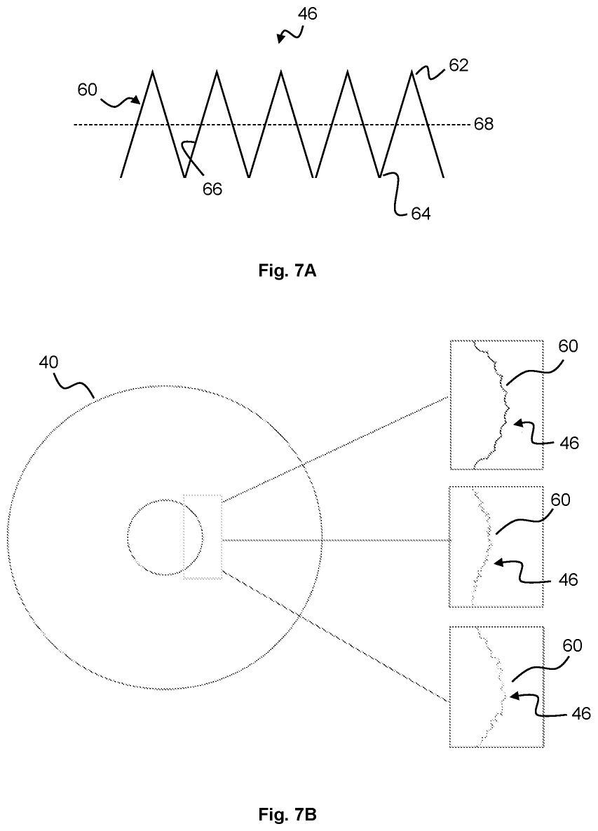

FIG. 7A is a sectional view of an inner perimeter 46 illustrated in FIG. 4A along line B-B. The inner perimeter 46 comprises a surface topography 60. The surface topography 60 is characterized by a plurality of crests 62 and troughs 64 with facets 66 disposed between the crests 62 and troughs 64. The crests 62 and troughs 64 are located a length from the mean plane 68.

FIG. 7B illustrates an ophthalmic pinhole prosthetic 40 and magnified views of various types of inner perimeters 46 according to the present disclosure. In the top magnified view, the inner perimeter 46 comprises a scalloped surface topography, as described herein. In the middle magnified view, the inner perimeter 46 comprises a jagged surface topography, as described herein, In the bottom magnified view, the inner perimeter 46 comprises an irregular surface topography, as described herein.

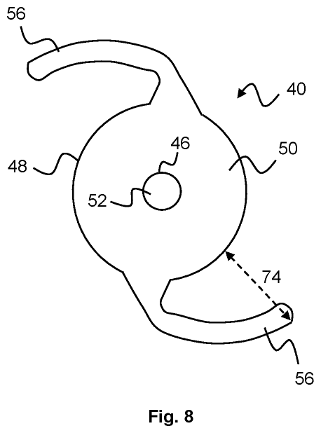

FIG. 8 is a plan view of an ophthalmic pinhole prosthetic 40. The ophthalmic pinhole prosthetic 40 comprises an annular portion 50 and an interior light transmitting portion 52. The annular portion 50 extends from the inner perimeter 46 to the outer perimeter 48. The ophthalmic pinhole prosthetic 40 comprises two J-shaped haptics 56 extending a length 74 radially from the outer perimeter 48 of the ophthalmic pinhole prosthetic 40. The haptics 56 extend from opposing sides of the outer perimeter 48. The haptics 56 include one end coupled to the outer perimeter 48 and end that is free.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The terms “generally” or “substantially” to describe angular measurements may mean about +/−10° or less, about +/−5° or less, or even about +/−1° or less. The terms “generally” or “substantially” to describe angular measurements may mean about +/−0.01° or greater, about +/−0.1° or greater, or even about +/−0.5° or greater. The terms “generally” or “substantially” to describe linear measurements, percentages, or ratios may mean about +/−10% or less, about +/−5% or less, or even about +/−1% or less. The terms “generally” or “substantially” to describe linear measurements, percentages, or ratios may mean about +/−0.01% or greater, about +/−0.1% or greater, or even about +/−0.5% or greater.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, from 20 to 80, or from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01, or 0.1 as appropriate. These are only examples of what is specifically intended and all possible combinations of numerical values between the lowest value and the highest value enumerated are to be considered to be expressly stated in this application in a similar manner. Unless otherwise stated, all ranges include both endpoints and all numbers between the endpoints.

As can be seen, the teaching of amounts expressed as “parts by weight” herein also contemplates the same ranges expressed in terms of percent by weight. Thus, an expression in the of a range in terms of “at least ‘x’ parts by weight of the resulting composition” also contemplates a teaching of ranges of same recited amount of “x” in percent by weight of the resulting composition.”

The term “consisting essentially of” to describe a combination shall include the elements, ingredients, components, or steps identified, and such other elements ingredients, components or steps that do not materially affect the basic and novel characteristics of the combination. The use of the terms “comprising” or “including” to describe combinations of elements, ingredients, components, or steps herein also contemplates embodiments that consist essentially of the elements, ingredients, components, or steps.

Plural elements, ingredients, components, or steps can be provided by a single integrated element, ingredient, component, or step. Alternatively, a single integrated element, ingredient, component, or step might be divided into separate plural elements, ingredients, components, or steps. The disclosure of “a” or “one” to describe an element, ingredient, component, or step is not intended to foreclose additional elements, ingredients, components, or steps.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

### REFERENCE NUMERALS

10 Eye
11 Aberration
12 Cornea
13 Lens
14 Iris
16 Pupil
18 Retina
20 Macula
22 Fovea
24 Optical axis
30 Light rays
32 Focal point
40 Ophthalmic pinhole prosthetic
42 Anterior surface
44 Posterior surface
46 Inner perimeter
48 Outer perimeter
50 Annular portion
52 Interior light transmitting portion
54 Surface modifications
56 Haptic
60 Surface topography
62 Crest
64 Trough
66 Facet
68 Mean plane
70 Diameter
72 Central axis
74 Length

What is claimed is:

1: An ophthalmic pinhole prosthetic comprising:
an annular portion that is optically opaque or at least partially optically opaque,
an inner perimeter including surface modifications configured to reduce or substantially eliminate the visually apparent effects of diffraction of light from the inner perimeter compared to an ophthalmic pinhole prosthetic without the surface modifications, and
an interior light transmitting portion located within the annular portion and functioning to allow passage of light to interact with a retina.

2: The ophthalmic pinhole prosthetic according to claim 1, wherein the inner perimeter circumscribes the interior light transmitting portion; and
wherein the interior light transmitting portion is an aperture or a portion of optically transparent material.

3: The ophthalmic pinhole prosthetic according to claim 2, wherein a diameter of the interior light transmitting portion is between about 0.05 mm and 3 mm.

4: The ophthalmic pinhole prosthetic according to claim 3, wherein the inner perimeter extends between an anterior surface and a posterior surface of the ophthalmic pinhole prosthetic.

5: The ophthalmic pinhole prosthetic according to claim 4, wherein a length of the inner perimeter, as measured between the anterior surface and the posterior surface, is between about 0.05 mm and 1 mm.

6: The ophthalmic pinhole prosthetic according to claim 4, wherein the inner perimeter slopes from the anterior surface to the posterior surface.

7: The ophthalmic pinhole prosthetic according to claim 4, wherein the inner perimeter includes a curvature.

8: The ophthalmic pinhole prosthetic according to claim 7, wherein the inner perimeter is convex or concave with respect to the interior light transmitting portion.

9: The ophthalmic pinhole prosthetic according to claim 1, wherein the ophthalmic pinhole prosthetic comprises one or more surface modifications on or proximate to the inner perimeter, the one or more surface modifications including one or more colors, one or more patterns of shapes, one or more surface topographies, one or more opacity gradients, or any combination thereof.

10: The ophthalmic pinhole prosthetic according to claim 9, wherein the surface modifications are between about 0.1 μm to 1000 μm in their largest dimension.

11: The ophthalmic pinhole prosthetic according to claim 1, wherein the ophthalmic pinhole prosthetic is insertable into an eye through an incision no greater than 3 mm in the largest dimension of the incision; and
wherein the largest dimension is the length between two opposing ends of the incision.

12: The ophthalmic pinhole prosthetic according to claim 11, wherein the ophthalmic pinhole prosthetic is flexible, foldable, or both; and
wherein the ophthalmic pinhole prosthetic is rollable into a roll having a major diameter no greater than 3 mm.

13: The ophthalmic pinhole prosthetic according to claim 1, wherein the ophthalmic pinhole prosthetic further comprises one or more haptics, which are configured to prevent the ophthalmic pinhole prosthetic from moving or rotating within an eye; and
wherein the one or more haptics are elongate projections that extend radially from an outer perimeter of the ophthalmic pinhole prosthetic.

14: The ophthalmic pinhole prosthetic according to claim 1, wherein an anterior surface of the ophthalmic pinhole prosthetic is convex, and a posterior surface of the ophthalmic pinhole prosthetic is concave; or both the anterior surface of the ophthalmic pinhole prosthetic and the posterior surface of the ophthalmic pinhole prosthetic are generally planar.

15: The ophthalmic pinhole prosthetic according to claim 1, wherein a thickness of the ophthalmic pinhole prosthetic, as measured between the anterior surface and the posterior surface, is between about 0.05 mm and 0.8 mm.

16: The ophthalmic pinhole prosthetic according to claim 15, wherein the thickness is generally uniform, varies radially from the inner perimeter to the outer perimeter, or both.

17: The ophthalmic pinhole prosthetic according to claim 1, wherein the ophthalmic pinhole prosthetic further comprises an outer perimeter that is radially distanced from the inner perimeter.

18: A method of fabricating the ophthalmic pinhole prosthetic according to claim 1, the method comprising:
locating an ophthalmic pinhole prosthetic substrate onto a support surface,
optionally locating a template in relation to the ophthalmic pinhole prosthetic substrate, the template having a plurality of masking regions and a plurality of pass-through regions, and
performing material addition and/or material removal on the ophthalmic pinhole prosthetic substrate to fabricate surface modifications.

19: The method according to claim 18, wherein the method comprises fabricating surface modifications on the ophthalmic pinhole prosthetic substrate concurrent with performing material addition and/or material removal to realize the final dimensions of the ophthalmic pinhole prosthetic.

20: The method according to claim 19, wherein the surface modifications include one or more colors, one or more patterns of shapes, one or more surface topographies, one or more opacity gradients, or any combination thereof; and
wherein the one or more surface modifications are located on or proximate to the inner perimeter.
